(19) United States
(12) Patent Application Publication
(10) Pub. No.: US 2022/0117521 A1
(43) Pub. Date: Apr. 21, 2022

(54) CLOUD SYSTEM OF NON-INVASIVE MEASURING BLOOD GLUCOSE

(71) Applicant: eTouch Medical Inc., New Taipei City (TW)

(72) Inventors: JENG-TAY LIN, New Taipei City (TW); SHENG-JIE LUO, New Taipei City (TW)

(21) Appl. No.: 17/074,633

(22) Filed: Oct. 20, 2020

(51) Int. Cl. A61B 5/145; G16H 50/50; A61B 5/00; G16H 50/30; G16H 50/70; G16H 40/67; H04Q 9/00; G01N 33/49; G06F 17/11; H04L 67/12; A61B 5/1477

(52) U.S. Cl. CPC A61B 5/14532; G16H 50/50; A61B 5/0022; A61B 5/742; G16H 50/30; G16H 50/70; H04Q 2209/43; H04Q 9/00; G01N 33/49; G06F 17/11; H04L 67/12; A61B 5/1477; G16H 40/67

(57) ABSTRACT

A cloud system of non-invasive measuring blood glucose is disclosed, and includes a non-invasive measuring blood glucose device, a mobile electronic device, and a cloud server for implementing a process of cloud measuring blood glucose. The non-invasive measuring blood glucose device is intended for a user to contact, and the mobile electronic device executing an application is in a non-contact manner connected to the non-invasive measuring blood glucose device. The cloud server is connected to the mobile electronic device. In particular, the non-invasive measuring blood glucose device generates a stimulating signal for the user to contact, and then induces a sensing signal respective of the stimulating signal, and the mobile electronic device receives and further transmits the sensing signal to the cloud server. The cloud server calculates and transmits blood glucose to the mobile electronic device for instantly displaying the blood glucose for the user.

### BACKGROUND OF THE INVENTION

### 1. Field of the Invention

The present invention generally relates to a cloud system of non-invasive measuring blood glucose, and more specifically to a cloud system of non-invasive measuring blood glucose provided with a non-invasive measuring blood glucose device, a mobile electronic device, and a cloud server for measuring blood glucose of an user by employing the non-invasive measuring blood glucose device to generate a sensing signal from the user, then the mobile electronic device executing an application to receive and transfer the sensing signal, the cloud server calculating blood glucose based on the sensing signal from the mobile electronic device to generate a blood glucose information comprising blood glucose, and the mobile electronic device displaying the blood glucose information, thereby readily and non-invasively measuring variation of blood glucose without sampling blood by penetrating the skin, avoiding any risk of infection caused by micro-organism, and greatly improving safety of measuring operation for ordinary people.

### 2. The Prior Arts

As well known, the amount of glucose in blood as so called blood glucose and usually comes from the intestine through digestion of the food, and is then transferred to every cell of the body as a primary source of energy for specific biochemical function. Blood glucose is thus one of crucial indications for active status and metabolism of human body. Only a narrow allowed range of blood glucose like 800-1200 mg/l is recommended for a healthy person.

It is confirmed that blood glucose dysfunction often and easily causes many chronic diseases such as hyperglycemia with constant high blood glucose and hypoglycemia with constantly low blood glucose. Hyperglycemia usually leads to diabetes, one of the most striking chronic diseases, and hypoglycemia makes syndromes like vertigo, lack of focus, and even shock.

For the patients suffering from diabetes, it is important to constantly take care of blood glucose, and appropriately inject insulin to reduce blood glucose to prevent healthy organs from adverse influence by high blood glucose,

Traditionally, blood glucose is measured by directly sampling blood by a lancet to penetrate human skin like a finger, and then dropping the sampled blood onto a test sheet or a test device. It is apparent that a wound caused by penetrating skin is easily infected by micro-organism like bacteria or virus, and human immunity may decrease because of constantly penetrating skin. Further, the wound needs to take a long time to close up.

Therefore, it is greatly needed to provide a new solution and system to non-invasive measure blood glucose provided with a non-invasive measuring blood glucose device, a mobile electronic device connected to the non-invasive measuring blood glucose device in a non-contact manner, and a cloud server connected to the mobile electronic device through a wireless network, the non-invasive measuring blood glucose device generating a sensing signal from the user, the mobile electronic device executing an application to receive and transfer the sensing signal, the cloud server calculating blood glucose based on the sensing signal from the mobile electronic device to generate a blood glucose information comprising blood glucose, the mobile electronic device displaying the blood glucose information, thereby readily measuring variation of blood glucose through a non-invasive manner without sampling blood by penetrating the skin, avoiding any risk of infection caused by micro-organism, greatly improving safety of measuring operation for ordinary people, and overcoming the above problems in the prior arts.

### SUMMARY OF THE INVENTION

The primary object of the present invention is to provide a cloud system of non-invasive measuring blood glucose comprising a non-invasive measuring blood glucose device, a mobile electronic device, and a cloud server for implementing a process of cloud measuring blood glucose. The non-invasive measuring blood glucose device is provided with a function of wireless communication, and generates a stimulating signal for a user to contact. The stimulating signal is substantially a square wave with a frequency within 100 and 500 Hz. The mobile electronic device is provided with a display screen, and executes an application (APP) to connect the non-invasive measuring blood glucose device in a non-contact manner to build up a wireless communication. The display screen is controlled by the application to show an operation frame serving as an operation interface for the user. In addition, the cloud server is away from and connected to the mobile electronic device through a wireless network.

Specifically, the operation of cloud measuring blood glucose comprises the following steps.

First, the non-invasive measuring blood glucose device waits for a preset period of waiting time after the user contacts the stimulating signal, then induces a sensing signal based on the stimulating signal, and transmits the sensing signal to the mobile electronic device. Thus, the sensing signal is responsive to the stimulating signal.

Then, the mobile electronic device receives and converts the sensing signal into a blood glucose sensing signal, and the blood glucose sensing signal is further transmitted to the cloud server, The cloud server employs the blood glucose sensing signal to perform a blood glucose calculation process to generate and transfer a blood glucose information to the mobile electronic device. The blood glucose information contains blood glucose of the user,

Finally, the mobile electronic device receives the blood glucose information, and the application executed by the mobile electronic device controls the operation frame of the display screen to display blood glucose of the user in the blood glucose information

Further, another object of the present invention is to provide a cloud system of non-invasive measuring blood glucose comprising a non-invasive measuring blood glucose device and a mobile electronic device for implementing a process of cloud measuring blood glucose. The non-invasive measuring blood glucose device with a function of wireless communication generates a stimulating signal for a user to contact. The stimulating signal is a square wave with a frequency within 100 and 500 Hz. The mobile electronic device is provided with a display screen, and executes an application to connect the non-invasive measuring blood glucose device in a non-contact manner to build up a wireless communication. The display screen is controlled by the application to show an operation frame serving as an operation interface for the user.

Specifically, the operation of cloud measuring blood glucose comprises the following steps.

First, the non-invasive measuring blood glucose device waits for a preset period of waiting time after the user contacts the stimulating signal, then induces a sensing signal based on the stimulating signal, and transmits the sensing signal to the mobile electronic device. Thus, the sensing signal is responsive to the stimulating signal.

Then, the mobile electronic device receives and converts the sensing signal into a blood glucose sensing signal, and the application executed by the mobile electronic device employs the blood glucose sensing signal to perform a blood glucose calculation process to generate and transfer blood glucose information. The blood glucose information contains blood glucose of the user, Finally, the application controls the operation frame of the display screen to display blood glucose of the user in the blood glucose information.

Therefore, the cloud system of non-invasive measuring blood glucose provides the non-invasive measuring blood glucose device connected to the electronic mobile device, the non-invasive measuring blood glucose device generates the sensing signal from the user, and the electronic mobile device or the cloud server connected to the electronic mobile device calculates blood glues of the user based on the sensing signal.

Overall, the cloud system of the present invention has a pretty simple structure and does not need any additional devices or connections to preliminarily and measure blood glucose in the non-contact manner, and instantly display blood glucose. It is convenient and practical for ordinary people to operate the present invention. The user does not only readily and fast measure blood glucose, but is also prevented from risk of infection by bacteria or virus through the wound of the skin for sampling blood. Particularly, the cloud system is suitable for a long term measurement of blood glucose.

### BRIEF DESCRIPTION OF THE DRAWINGS

The present invention can be understood in more detail by reading the subsequent detailed description in conjunction with the examples and references made to the accompanying drawings, wherein:

FIG. 1 is a view showing the cloud system of non-invasive measuring blood glucose according to the first embodiment of the present invention;

FIG. 2 is a view showing the input electrode unit and the output electrode unit of the system according to the first embodiment of the present invention;

FIG. 3 is another view showing the input electrode unit and the output electrode unit of the system according to the first embodiment of the present invention; and

FIG. 4 is a view showing the cloud system of non-invasive measuring blood glucose according to the second embodiment of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) show and depict only the preferred embodiments of the invention and shall not be considered as limitations to the scope of the present invention. Modifications of the shape of the present invention shall too be considered to be within the spirit of the present invention.

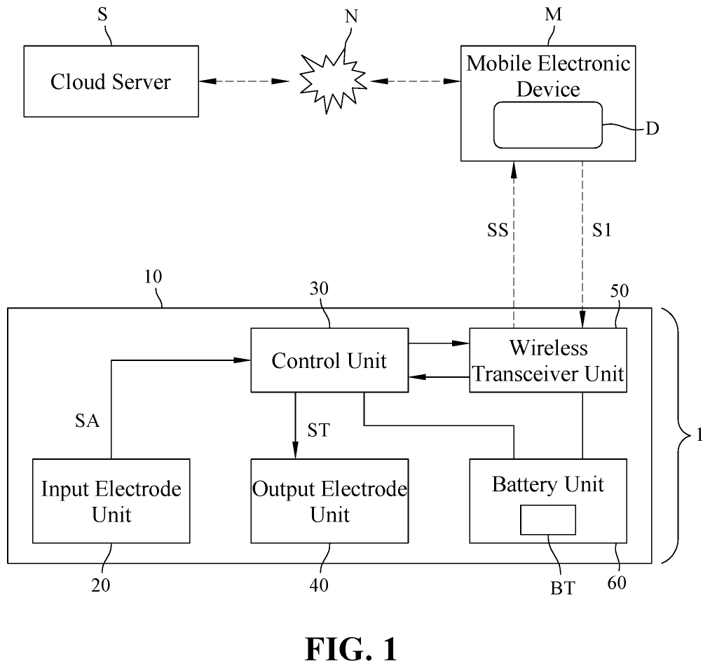

Please refer to FIG. 1 illustrating the cloud system of non-invasive measuring blood glucose according to the first embodiment of the present invention. As shown in FIG. 1, the cloud system of non-invasive measuring blood glucose generally comprises a non-invasive measuring blood glucose device 1, a mobile electronic device M, and a cloud server S for implementing a process of cloud measuring blood glucose. The non-invasive measuring blood glucose device 1 is provided with a function of wireless communication, and generates a stimulating signal ST for a user to contact. The stimulating signal ST is substantially a square wave with a frequency within 100 and 500 Hz. The mobile electronic device M is provided with a display screen D, and executes an application (APP) to connect the non-invasive measuring blood glucose device 1 in a non-contact manner to build up a wireless communication. The display screen D is controlled by the application to show an operation frame serving as an operation interface for the user. In addition, the cloud server S is away from and connected to the mobile electronic device M through a wireless network N.

For example, the mobile electronic device M comprises at least one of a smart phone and a tablet computer, the non-contact manner comprises at least one of Bluetooth, wireless fidelity (Wi-Fi), near field communication (NFC), and Zigbee, and the wireless network comprises at least one of local wireless network, a third generation (3G) mobile communication network, a fourth generation (4G) mobile communication network, and a fifth generation (5G) mobile communication network.

Specifically, the operation of cloud measuring blood glucose comprises the steps as follows.

First, the non-invasive measuring blood glucose device 1 waits for a preset period of waiting time like 0.6 to 1.2 seconds after the user contacts the stimulating signal ST, then induces a sensing signal SS based on the stimulating signal ST, and transmits the sensing signal SS to the mobile electronic device M. Thus, the sensing signal SS is responsive to the stimulating signal ST. Theoretically, the skin intrinsically possesses various electrical signals respective of biochemical reactions of skin issue, flowing blood, and electrical pulse of neuron, as well as a large equivalent capacitor, and when the user contacts the outer surface of the non-invasive measuring blood glucose device 1, the sensing signal SS responsive to the stimulating signal ST is induced and generated. Further, the sensing signal SS basically responds to electrical property of body issues, particularly biochemical matters flowing through blood vessels such as glucose, urine acid, antibodies, blood corpuscles, Thus, the present invention specifically limits the frequency of the stimulating signal ST to range from 100 to 500 Hz to make the sensing signal SS exclusively respond to blood glucose.

Next, the mobile electronic device M receives and converts the sensing signal SS into a blood glucose sensing signal, and the blood glucose sensing signal is further transmitted to the cloud server S, The cloud server S employs the blood glucose sensing signal to perform a blood glucose calculation process to generate and transfer a blood glucose information to the mobile electronic device M. The blood glucose information contains blood glucose of the user, Finally, the mobile electronic device M receives the blood glucose information, and the application executed by the mobile electronic device M controls the operation frame of the display screen D to display blood glucose of the user in the blood glucose information.

In short, the user just contacts the non-invasive measuring blood glucose device 1 and then views blood glucose on the display screen D of the mobile electronic device M through the application in collocation with the cloud server S such that the overall operation for blood glucose measurement is quite simple and convenient for ordinary people with little practice.

More specifically, the user just contacts the non-invasive measuring blood glucose device 1 comprises a case 10, an input electrode unit 20, a control unit 30, an output electrode unit 40, a wireless transceiver unit 50, and a battery unit 60, and the case 10 is provided with electrical insulation and water-proof, as well as an accommodating space. The control unit 30, the wireless transceiver unit 50, and the battery unit 60, are accommodated in the accommodating space, and the input electrode unit 20 and the output electrode unit 40 are provided on the outer surface of the case 10 like the front surface for the user to contact. In particular, the input electrode unit 20 and the output electrode unit 40 are not connected or contact each other. In addition, the battery unit 60 comprises at least one battery BT to supply power for the control unit 30 and the wireless transceiver unit 50 to operate.

Further, the input electrode unit 20 and the output electrode unit 40 are electrically connected to the control unit 30, and made of electrically conductive material, and have a thin sheet shape. When the user contacts the input electrode unit 20, the input electrode unit 20 waits for a preset period of waiting time, and then induces and transmits a sensing input signal SA to for the control unit 30. The control unit 30 subsequently filters, amplifies, and converts the sensing input signal SA into the sensing signal SS, and the sensing signal SS is then transmitted. The wireless transceiver unit 50 is electrically connected to the control unit 30 for receiving and transmitting the sensing signal SS to the mobile electronic device M. It should be noted that the sensing signal SS is substantially of a digital signal through a process of analog to digital conversion (ADC) performed by the control unit 30. For instance, the sensing signal SS ranges from 0 to 4095 for 12 bit ADC and 3.3V operation power, wherein 0 refers to 0V, and 4095 refers to 3.3V. The above process of ADC is commonly used in the prior arts, and not described in detail hereinafter.

The control unit 30 generates and transmits the stimulating signal ST in an automatic manner or a passive manner. For the automatic manner, the control unit 30 constantly generates the stimulating signal ST without any external control. For the passive manner, the control unit 30 first receives an external stimulating signal S1, and then generates the stimulating signal ST based on the external stimulating signal S1. In other words, the stimulating signal ST is only generated upon the external stimulating signal S1 received. For example, the external stimulating signal S1 is generated and transmitted by the mobile electronic device M to the control unit 30 though the wireless transceiver unit 50. Or alternatively, the external stimulating signal S1 is first generated and transmitted by the cloud server S to the mobile electronic device M, and then further transmitted from the mobile electronic device M to the control unit 30 though the wireless transceiver unit 50. The control unit 30 transmits the stimulating signal ST to the output electrode unit 40 for the user to contact so as to cause the input electrode unit 20 to induce the sensing input signal SA as mentioned above.

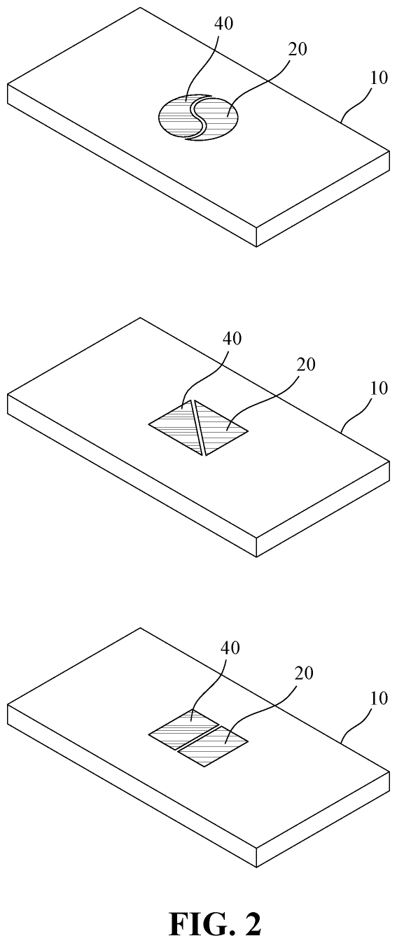

For instance, the input electrode unit 20 and the output electrode unit 40 are provided in a preset region as shown in FIG. 2. The preset region has an area specifically smaller than an area of a front surface of the finger. For here, the front surface of the finger refers to a surface of the finger with a fingerprint, and the finger comprises one of a thumb, a forefinger, a middle finger, a ring finger, and a little finger. Particularly, each of the input electrode unit 20 and the output electrode unit 40 comprises at least one pattern like tai-chi pattern, triangular pattern, rectangular pattern, or half-moon pattern. However, FIG. 2 illustrates that the input electrode unit 20 and the output electrode unit 40 has only one tai-chi pattern, one triangular pattern, or one rectangular pattern as an exemplary case for clearly describing the aspects of the present invention.

Further, the blood glucose calculation process performed by the cloud server S is in collocation with the input electrode unit 20 and the output electrode unit 40 as shown in FIG. 2 and comprises the steps as described below.

First, the cloud server S samples and collects the sensing signal SS, then averages eight to twenty successive sensing signals SS to calculate an arithmetic mean signal, and the arithmetic mean signal is compared with a preset noise threshold until the arithmetic mean signal is not larger than the preset noise threshold. Specifically, the arithmetic mean signal not larger than the preset noise threshold is served as an effective sensing signal, and the preset noise threshold is a real number within 300 and 500. For here, the arithmetic mean signal larger than the preset noise threshold is discarded as ineffective signal, and it is different from tradition treatment for noise which is usually smaller than the effective signal. The reason is that the sensing signal SS is induced after the user contacts the input electrode unit 20 and the output electrode unit 40 like contacting with one finger, and the sensing signal SS is easily interfered by external environment and abruptly increases up to a considerable value, that is, a peak, lasting for a period of time such that the arithmetic mean signal becomes much larger and distorted, failing to sincerely represent blood glucose.

Then, the above effective sensing signal is taken as a finger signal.

Subsequently, the finger signal is employed to calculate a finger feedback signal by an equation specified by A1_ratio=para_1*A1_m_Ave+para_2, where A1_ratio indicates the finger feedback signal, and para_1 is a first parameter as a real number within 0.055 and 0.065, para_2 is a second parameter as a real number within 25.31 and 25.51. Further, A1_m_Ave is an average of A1_m, A1_m is A1_ave not larger than a value specified as 600 to 1500 and served as a stable feedback signal out of an extreme range, where A1_ave is a value of 100 average signals, and each average signal is an average of 10 successive finger signals.

Finally, the finger feedback signal is used to calculate blood glucose, blood glucose is further incorporated into the blood glucose information, and the application controls the operation frame displayed on the display screen D to provide an operation mode to the user to select. The operation mode comprises at least one of an empty stomach mode, a meal after mode, a normal healthy mode, a diabetes pre-stage mode, and a diabetes mode. The above blood glucose is indicated by GLU and calculated by an equation as GLU=para_3*(((para_4−A1_ratio)/Para_6)−para_5), where para_3 is a third parameter, para_4 is a fourth parameter, para_5 is a fifth parameter, and para_6 is a sixth parameter. It is preferred that para_3 is a real number within 2.8 and 3.9 for the normal healthy mode, within 2.86 and 5.58 for the diabetes pre-stage mode, and within 4.68 and 19.5 for the diabetes mode, para_4 is a real number within 60 and 70 for the empty stomach mode, and within 71 and 80 for the meal after mode, para_5 is a real number within 0.03 and 0.06, and para_6 is a real number within 10.211 and 10.519. More specifically, the above equation for calculating blood glucose is based on a big data through statistically recursion analysis, and the big data generally covers healthy persons and sick people for a wide range of ages, nations, occupations, and so forth. In short, blood glucose and A1_ratio are correlated in a linear form, and the data for blood glucose measured by the cloud system of the present invention is compared with blood glucose by traditional methods, as a result of accuracy of particularly up to 94%.

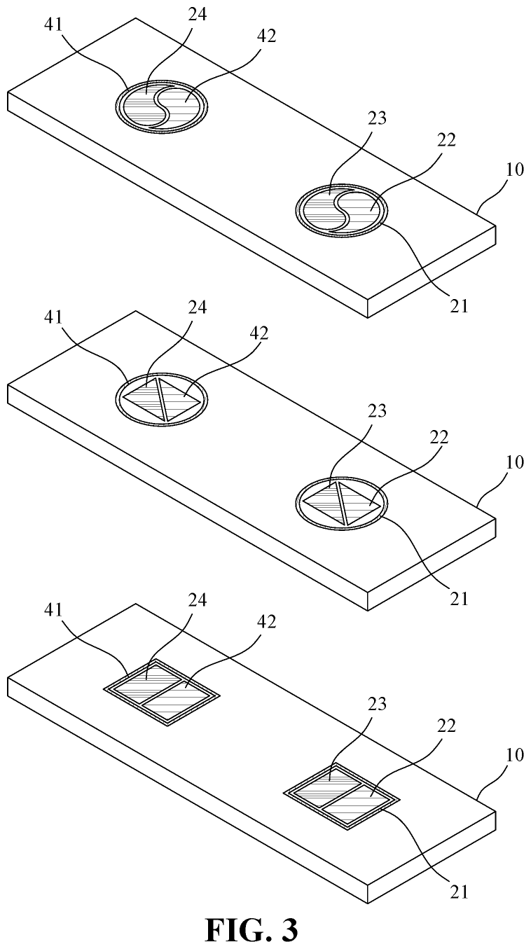

Or alternatively, another illustrative example for the input electrode unit 20 and the output electrode unit 40 is shown in FIG. 3. The input electrode unit 20 comprises a first input electrode 21, a second input electrode 22, a third input electrode 23, and a fourth input electrode 24, and the output electrode unit 40 comprises a first output electrode 41 and a second output electrode 42. Specifically, the first output electrode 41 has a ring shape with a central hollow, the second output electrode 42 has a shape of a specific pattern like tai-chi pattern, triangular pattern, rectangular pattern, or half-moon pattern, and the second output electrode 42 is provided in the ring shape of the first output electrode 41. Further, the first input electrode 21 has a ring shape with a central hollow, and each of the second input electrode 22, the third input electrode 23, and the fourth input electrode 24 has a shape of a specific pattern like tai-chi pattern, triangular pattern, rectangular pattern, or half-moon pattern. The second input electrode 22 and the third input electrode 23 are provided in the ring shape of the first input electrode 21, and the first input electrode 21, the second input electrode 22, and the third input electrode 23 are not in contact with each other. In particular, the fourth input electrode 24 is provided in the ring shape of the first output electrode 41, and the fourth input electrode 24, the first output electrode 41, and the second output electrode 42 are not in contact with each other.

Moreover, each of the size of the ring shape of the first output electrode 41 and the size of the ring shape of the first input electrode 21 is equal to or larger than a contact area of a finger tip of the finger of the user in contact with the input electrode unit 20 or the output electrode unit 40.

In addition, the blood glucose calculation process performed by the cloud server S is in collocation with the input electrode unit 20 and the output electrode unit 40 as shown in FIG. 3 and comprises the steps as described below.

First, the cloud server S samples and collects the sensing signal SS, then averages eight to twenty successive sensing signals SS to calculate an arithmetic mean signal, and the arithmetic mean signal is compared with a preset noise threshold until the arithmetic mean signal is not larger than the preset noise threshold. Specifically, the arithmetic mean signal not larger than the preset noise threshold is served as an effective sensing signal, and the preset noise threshold is a real number within 300 and 500.

Next, the effective sensing signal is divided into a first finger signal and a second finger signal. The first finger signal is served as a signal from a first finger of the user in contact with the first input electrode 21, the second input electrode 22, and the third input electrode 23, and the second finger signal is served as a signal from a second finger of the user in contact with the first output electrode 41, the second output electrode 42, and the forth input electrode 24. Specifically, the first finger is a thumb or a forefinger of a right hand of the user, and the second finger is a thumb or a forefinger of a left hand of the user. Or alternatively, the first finger is the thumb or the forefinger of the left hand, and the second finger is the thumb or the forefinger of the right hand.

Subsequently, the first finger signal is employed to calculate a first finger feedback signal according to an equation specified by A1_ratio=P1*A1_m_ave+P2, where A1_ratio indicates the first finger feedback signal, P1 is a first parameter as a real number within 0.05 and 0.08, and P2 is a second parameter as a real number within 21.05 and 35.34. Further, A1_m_Ave is an average of A1_m, A1_m is A1_ave not larger than a value specified as 600 to 1500 and served as a stable feedback signal out of an extreme range, and A1_ave is a value of 100 average signals, each average signal being an average of 10 successive first finger signals.

Then, the second finger signal is used to calculate a second finger feedback signal indicated by A2_m_Ave. Specifically, A2_m_Ave is an average of A2_m, A2_m is A2_ave not larger than a value specified as 900 to 1800 and served as a stable feedback signal out of an extreme range, and A2_ave is a value of 100 average signals, each average signal being an average of 10 successive second finger signals.

Finally, the first finger feedback signal and the second finger feedback signal are employed to calculate blood glucose, then blood glucose is incorporated into the blood glucose information, and the application controls the operation frame displayed on the display screen D to provide an operation mode to the user to select. For example, the operation mode comprises at least one of an empty stomach mode, a meal after mode, a normal healthy mode, a diabetes pre-stage mode, and a diabetes mode. More specifically, the blood glucose is indicated by GLU and calculated by an equation as GLU=P3*(A2_m_ave/P4)−P5)*(((P6−A1_ratio)/10.238)−P5)*P7, where P3 is a third parameter, P4 is a fourth parameter, P5 is a fifth parameter, P6 is a sixth parameter, and P7 is a seventh parameter. It is preferred that P3 is a real number within 0.8 and 1.0 for the normal healthy mode, within 1.1 and 1.5 for the diabetes pre-stage mode, and within 1.8 and 5.0 for the diabetes mode, P4 is a real number within 210 and 220 for the empty stomach mode, and within 200 and 210 for the meal after mode, P5 is a real number within 0.03 and 0.06, P6 is a real number within 60 and 70 for the empty stomach mode, and within 71 and 80 for the meal after mode, and P7 is a real number indicating percentage within 3%-15%. Accordingly, the above equation for calculating GLU is based on a big data through statistically recursion analysis.

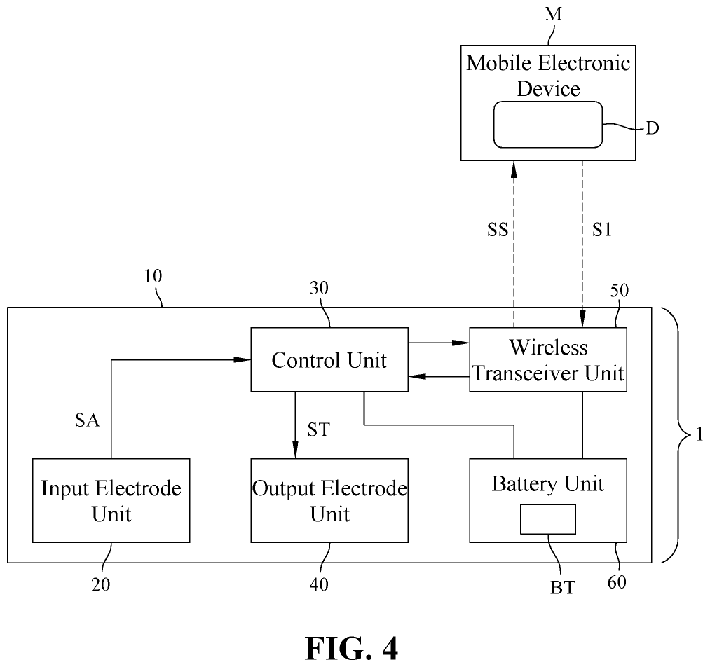

Further refer to FIG. 4 illustrating the cloud system of non-invasive measuring blood glucose according to the second embodiment of the present invention. As shown in FIG. 4, the cloud system of the second embodiment comprises a non-invasive measuring blood glucose device 1 and a mobile electronic device M for implementing a process of cloud measuring blood glucose.

It should be noted that the cloud system of the second embodiment is similar to the first embodiment as shown in FIG. 1, and the primary difference is that the second embodiment does not comprise the cloud server. In other words, the non-invasive measuring blood glucose device 1 of the second embodiment is identical to the non-invasive measuring blood glucose device 1 of the first embodiment. Further, the mobile electronic device M of the second embodiment substantially implements the function of the cloud server to calculate blood glucose. Thus, the operation of the non-invasive measuring blood glucose device 1 is not described, and only the mobile electronic device M is further illustrated hereinafter.

Specifically, the mobile electronic device M is accordingly connected to the non-invasive measuring blood glucose device 1 through the application for wireless communication, and the operation frame is displayed on the display screen D as the operation interface. However, the application executed by the mobile electronic device M implements the operation of the cloud server. For example, the mobile electronic device M is directly intended to generate and transmit the external stimulating signal S1 to the non-invasive measuring blood glucose device 1, and the blood glucose information is generated by the application.

Further, the operation of cloud measuring blood glucose comprises the steps as below.

First, the non-invasive measuring blood glucose device 1 waits for a preset period of waiting time after the user contacting the stimulating signal ST, then induces a sensing signal SS based on the stimulating signal ST, and transmits the sensing signal SS to the mobile electronic device M. That is, the sensing signal SS is responsive to the stimulating signal ST.

Then, the mobile electronic device M receives and converts the sensing signal SS into a blood glucose sensing signal through the application, and the blood glucose sensing signal is further employed to perform a blood glucose calculation process to generate a blood glucose information. Accordingly, the blood glucose information contains blood glucose of the user. Finally, the application of the mobile electronic device M controls the operation frame of the display screen D to display blood glucose in the blood glucose information for the user to reference.

In contrast to the first embodiment, the mobile electronic device M of the second embodiment employs the application to generate the external stimulating signal S1, and also performs the blood glucose calculation process to generate a blood glucose information containing blood glucose of the user, and the overall structure of the second embodiment is thus more practical and beneficial to industrial utility. In particular, only communication between the non-invasive measuring blood glucose device 1 and the mobile electronic device M like short range communication is needed to implement the function of blood glucose measurement. As a result, interference caused by poor station signal is prevented in the second embodiment, especially for a remote site or a closed indoor environment.

Therefore, one of the aspects provided by the present invention is to implement the function of non-invasive measuring blood glucose device through the non-invasive measuring blood glucose device, the mobile electronic device, and the cloud server. The non-invasive measuring blood glucose device generates the sensing signal from the user, the mobile electronic device executes the application to receive and transfer the sensing signal, the cloud server calculates blood glucose based on the sensing signal to generate the blood glucose information comprising blood glucose, and the mobile electronic device displays the blood glucose information for the user to reference, It is convenient for ordinary people to readily and constantly measure variation of blood glucose without penetrating the skin for sampling blood, and any risk of infection caused by micro-organism is prevented as well as greatly improving safety of measuring operation.

In addition, another aspect is that the cloud server is replaced by the mobile electronic device wirelessly connected to the non-invasive measuring blood glucose device. The user just contacts the non-invasive measuring blood glucose device, and readily views the result of blood glucose on the display screen of the mobile electronic device. In other words, the application of the mobile electronic device implements the process of calculating blood glucose and shows the blood glucose information. It is feasible for the user to readily measure blood glucose by just carrying the non-invasive measuring blood glucose device for sensing the finger signal by contact or touch, and the mobile electronic device for calculating and displaying blood glucose. Furthermore, the application can be easily maintained and updated to increase accuracy of measurement according to current health status for individuals.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A cloud system of non-invasive measuring blood glucose for implementing an operation of cloud measuring blood glucose, comprising:
a non-invasive measuring blood glucose device with a function of wireless communication, generating a stimulating signal for a user to contact, the stimulating signal being a square wave with a frequency within 100 and 500 Hz;
a mobile electronic device provided with a display screen, executing an application (APP) to connect the non-invasive measuring blood glucose device in a non-contact manner to build up a wireless communication, the display screen showing an operation frame serving as an operation interface for the user; and
a cloud server away from and connected to the mobile electronic device through a wireless network,
wherein the operation of cloud measuring blood glucose comprising steps of:
the non-invasive measuring blood glucose device waiting for a preset period of waiting time after the user contacting the stimulating signal, then inducing a sensing signal based on the stimulating signal, and transmitting the sensing signal to the mobile electronic device, the sensing signal responsive to the stimulating signal;
the mobile electronic device receiving the sensing signal, converting the sensing signal into a blood glucose sensing signal, and transmitting the blood glucose sensing signal to the cloud server;
the cloud server receiving and employing the blood glucose sensing signal to perform a blood glucose calculation process to generate and transfer a blood glucose information to the mobile electronic device, the blood glucose information containing blood glucose of the user;
the mobile electronic device receiving the blood glucose information; and
the application executed by the mobile electronic device controlling the operation frame of the display screen to display blood glucose of the user in the blood glucose information.

2. The cloud system as claimed in claim 1, wherein the mobile electronic device comprises at least one of a smart phone and a tablet computer, the non-contact manner comprises at least one of bluetooth, wireless fidelity (Wi-Fi), near field communication (NFC), and Zigbee, the wireless network comprises at least one of local wireless network, a third generation (3G) mobile communication network, a fourth generation (4G) mobile communication network, and a fifth generation (5G) mobile communication network, and the preset period of waiting time is 0.6 to 1.2 seconds.

3. The cloud system as claimed in claim 1, wherein the non-invasive measuring blood glucose device comprising:
a case with electrical insulation and water-proof, having an accommodating space;
an input electrode unit provided on an outer surface of the case, formed of an electrically conductive material, having a thin sheet shape for the user to contact, inducing and transmitting a sensing input signal after the preset period of waiting time when the user contacting the input electrode unit and the stimulating signal;
a control unit provided in the accommodating space, electrically connected to the input electrode unit, receiving, filtering, amplifying, and converting the sensing input signal into the sensing signal, generating and transmitting the stimulating signal in an automatic manner or a passive manner;
an output electrode unit provided on the outer surface of the case, not in contact with the input electrode unit, formed of the electrically conductive material, having a thin sheet shape, electrically connected to the control unit for receiving the stimulation signal for the user to contact;
a wireless transceiver unit provided in the accommodating space, electrically connected to the control unit for receiving and transmitting the sensing signal to the mobile electronic device; and
a battery unit provided in the accommodating space, comprising at least one of battery for supply electric power to the control unit and the wireless transceiver unit for operation, the passive manner implemented by the control unit receiving an external stimulating signal from the mobile electronic device, the external stimulating signal generated and transmitted by the mobile electronic device to the control unit though the wireless transceiver unit, or alternatively, the external stimulating signal generated and transmitted by the cloud server to the mobile electronic device, the external stimulating signal further transmitted from the mobile electronic device to the control unit though the wireless transceiver unit.

4. The cloud system as claimed in claim 3, wherein the input electrode unit and the output electrode unit are provided in a sensing area of the outer surface of the case, the sensing area has an area smaller than a positive area of a finger of the user for contacting the input electrode unit and the output electrode unit, the positive area refers to a surface of the finger with a fingerprint, the finger comprising one of a thumb, a forefinger, a middle finger, a ring finger, and a little finger, each of the input electrode unit and the output electrode unit comprises at least one pattern, and the blood glucose calculation process performed by the cloud server comprises:
sampling and collecting the sensing signal;
averaging eight to twenty successive sensing signals to calculate an arithmetic mean signal and comparing the arithmetic mean signal with a preset noise threshold until the arithmetic mean signal is not larger than the preset noise threshold, the arithmetic mean signal not larger than the preset noise threshold served as an effective sensing signal, the preset noise threshold being a real number within 300 and 500;
taking the effective sensing signal as a finger signal;
calculating a finger feedback signal based on the finger signal by an equation specified by A1_ratio=para_1*A1_m_Ave+para_2, A1_ratio indicating the finger feedback signal, para_1 being a first parameter as a real number within 0.055 and 0.065, para_2 being a second parameter as a real number within 25.31 and 25.51, A1_m_Ave being an average of A1_m, A1_m being A1_ave not larger than a value specified as 600 to 1500 and served as a stable feedback signal out of an extreme range, A1_ave being a value of 100 average signals, each average signal being an average of 10 successive finger signals; and
calculating blood glucose based on the finger feedback signal, incorporating blood glucose into the blood glucose information, and the application controlling the operation frame displayed on the display screen to provide an operation mode to the user to select, the operation mode comprising at least one of an empty stomach mode, a meal after mode, a normal healthy mode, a diabetes pre-stage mode, and a diabetes mode, the blood glucose indicated by GLU and calculated by an equation as GLU=para_3*(((para_4−A1_ratio)/Para_6)−para_5), para_3 being a third parameter, para_4 being a fourth parameter, para_5 being a fifth parameter, para_6 being a sixth parameter, para_3 as a real number within 2.8 and 3.9 for the normal healthy mode, within 2.86 and 5.58 for the diabetes pre-stage mode, and within 4.68 and 19.5 for the diabetes mode, para_4 as a real number within 60 and 70 for the empty stomach mode, and within 71 and 80 for the meal after mode, para_5 as a real number within 0.03 and 0.06, para_6 as a real number within 10.211 and 10.519.

5. The cloud system as claimed in claim 3, wherein the output electrode unit comprises a first output electrode and a second output electrode, the input electrode unit comprises a first input electrode, a second input electrode, a third input electrode, and a fourth input electrode, the first output electrode has a ring shape with a central hollow, the second output electrode has a shape of a pattern, the second output electrode is provided in the ring shape of the first output electrode, the first input electrode has a ring shape with a central hollow, each of the second input electrode, the third input electrode, and the fourth input electrode has a shape of a pattern, the second input electrode and the third input electrode are provided in the ring shape of the first input electrode, the first input electrode, the second input electrode and the third input electrode are not in contact with each other, the fourth input electrode is provided in the ring shape of the first output electrode, the fourth input electrode, the first output electrode, and the second output electrode are not in contact with each other, each of a size of the ring shape of the first output electrode and a size of the ring shape of the first input electrode is equal to or larger than a contact area of a finger tip of the finger of the user in contact with the input electrode unit or the output electrode unit, and the blood glucose calculation process performed by the cloud server comprises:
sampling and collecting the sensing signal;
averaging eight to twenty successive sensing signals to calculate an arithmetic mean signal and comparing the arithmetic mean signal with a preset noise threshold until the arithmetic mean signal is not larger than the preset noise threshold, the arithmetic mean signal not larger than the preset noise threshold served as an effective sensing signal, the preset noise threshold being a real number within 300 and 500;
dividing the effective sensing signal into a first finger signal and a second finger signal, the first finger signal served as a signal from a first finger of the user in contact with the first input electrode, the second input electrode, and the third input electrode, the second finger signal served as a signal from a second finger of the user in contact with the first output electrode, the second output electrode, and the forth input electrode, the first finger is a thumb or a forefinger of a right hand of the user, and the second finger is a thumb or a forefinger of a left hand of the user, or alternatively, the first finger is the thumb or the forefinger of the left hand, and the second finger is the thumb or the forefinger of the right hand;
calculating a first finger feedback signal based on the first finger signal by an equation specified by A1_ratio=P1*A1_m_ave+P2, A1_ratio indicating the first finger feedback signal, P1 being a first parameter as a real number within 0.05 and 0.08, P2 being a second parameter as a real number within 21.05 and 35.34, A1_m_Ave being an average of A1_m, A1_m being A1_ave not larger than a value specified as 600 to 1500 and served as a stable feedback signal out of an extreme range, A1_ave being a value of 100 average signals, each average signal being an average of 10 successive first finger signals;
calculating a second finger feedback signal based on the second finger signal by A2_m_Ave, A2_m_Ave being an average of A2_m, A2_m being A2_ave not larger than a value specified as 900 to 1800 and served as a stable feedback signal out of an extreme range, A2_ave being a value of 100 average signals, each average signal being an average of 10 successive second finger signals; and
calculating blood glucose based on the first finger feedback signal and the second finger feedback signal, incorporating blood glucose into the blood glucose information, and the application controlling the operation frame displayed on the display screen to provide an operation mode to the user to select, the operation mode comprising at least one of an empty stomach mode, a meal after mode, a normal healthy mode, a diabetes pre-stage mode, and a diabetes mode, the blood glucose indicated by GLU and calculated by an equation as GLU=P3*(A2_m_ave/P4)−P5)*(((P6−A1_ratio)/10.238)−P5)*P7, P3 being a third parameter, P4 being a fourth parameter, P5 being a fifth parameter, P6 being a sixth parameter, P7 being a seventh parameter, P3 as a real number within 0.8 and 1.0 for the normal healthy mode, within 1.1 and 1.5 for the diabetes pre-stage mode, and within 1.8 and 5.0 for the diabetes mode, P4 as a real number within 210 and 220 for the empty stomach mode, and within 200 and 210 for the meal after mode, P5 as a real number within 0.03 and 0.06, P6 as a real number within 60 and 70 for the empty stomach mode, and within 71 and 80 for the meal after mode, P7 as a real number indicating percentage within 3%-15%.

6. A cloud system of non-invasive measuring blood glucose for implementing an operation of cloud measuring blood glucose, comprising:
a non-invasive measuring blood glucose device with a function of wireless communication, generating a stimulating signal for a user to contact, the stimulating signal being a square wave with a frequency within 100 and 500 Hz; and
a mobile electronic device provided with a display screen, executing an application (APP) to connect the non-invasive measuring blood glucose device in a non-contact manner to build up a wireless communication, the display screen showing an operation frame serving as an operation interface for the user,
wherein the operation of cloud measuring blood glucose comprising steps of:
the non-invasive measuring blood glucose device waiting for a preset period of waiting time after the user contacting the stimulating signal, then inducing a sensing signal based on the stimulating signal, and transmitting the sensing signal to the mobile electronic device, the sensing signal responsive to the stimulating signal;
the mobile electronic device receiving the sensing signal and converting the sensing signal into a blood glucose sensing signal;
the application executed by the mobile electronic device employing the blood glucose sensing signal to perform a blood glucose calculation process to generate a blood glucose information, the blood glucose information containing blood glucose of the user; and
the application controlling the operation frame of the display screen to display blood glucose of the user in the blood glucose information.

7. The cloud system as claimed in claim 6, wherein the mobile electronic device comprises at least one of a smart phone and a tablet computer, the non-contact manner comprises at least one of bluetooth, wireless fidelity (Wi-Fi), near field communication (NFC), and Zigbee, and the preset period of waiting time is 0.6 to 1.2 second.

8. The cloud system as claimed in claim 6, wherein the non-invasive measuring blood glucose device comprising:
a case with electrical insulation and water-proof, having an accommodating space;
an input electrode unit provided on an outer surface of the case, formed of an electrically conductive material, having a thin sheet shape for the user to contact, inducing and transmitting a sensing input signal after the preset period of waiting time when the user contacting the input electrode unit and the stimulating signal;
a control unit provided in the accommodating space, electrically connected to the input electrode unit, receiving, filtering, amplifying, and converting the sensing input signal into the sensing signal, generating and transmitting the stimulating signal in an automatic manner or a passive manner;
an output electrode unit provided on the outer surface of the case, not in contact with the input electrode unit, formed of the electrically conductive material, having a thin sheet shape, electrically connected to the control unit for receiving the stimulation signal for the user to contact;
a wireless transceiver unit provided in the accommodating space, electrically connected to the control unit for receiving and transmitting the sensing signal to the mobile electronic device; and
a battery unit provided in the accommodating space, comprising at least one of battery for supply electric power to the control unit and the wireless transceiver unit for operation, the passive manner implemented by the control unit receiving an external stimulating signal from the mobile electronic device, the external stimulating signal generated and transmitted by the mobile electronic device to the control unit though the wireless transceiver unit.

9. The cloud system as claimed in claim 8, wherein the input electrode unit and the output electrode unit are provided in a sensing area of the outer surface of the case, the sensing area has an area smaller than a positive area of a finger of the user for contacting the input electrode unit and the output electrode unit, the positive area refers to a surface of the finger with a fingerprint, the finger comprising one of a thumb, a forefinger, a middle finger, a ring finger, and a little finger, each of the input electrode unit and the output electrode unit comprises at least one pattern, and the blood glucose calculation process performed by the mobile electronic device comprises:
sampling and collecting the sensing signal;
averaging eight to twenty successive sensing signals to calculate an arithmetic mean signal and comparing the arithmetic mean signal with a preset noise threshold until the arithmetic mean signal is not larger than the preset noise threshold, the arithmetic mean signal not larger than the preset noise threshold served as an effective sensing signal, the preset noise threshold being a real number within 300 and 500;
taking the effective sensing signal as a finger signal;
calculating a finger feedback signal based on the finger signal by an equation specified by A1_ratio=para_1*A1_m_Ave+para_2, A1_ratio indicating the finger feedback signal, para_1 being a first parameter as a real number within 0.055 and 0.065, para_2 being a second parameter as a real number within 25.31 and 25.51, A1_m_Ave being an average of A1_m, A1_m being A1_ave not larger than a value specified as 600 to 1500 and served as a stable feedback signal out of an extreme range, A1_ave being a value of 100 average signals, each average signal being an average of 10 successive finger signals; and
calculating blood glucose based on the finger feedback signal, incorporating blood glucose into the blood glucose information, and the application controlling the operation frame displayed on the display screen to provide an operation mode to the user to select, the operation mode comprising at least one of an empty stomach mode, a meal after mode, a normal healthy mode, a diabetes pre-stage mode, and a diabetes mode, the blood glucose indicated by GLU and calculated by an equation as GLU=para_3*(((para_4−A1_ratio)/Para_6)−para_5), para_3 being a third parameter, para_4 being a fourth parameter, para_5 being a fifth parameter, para_6 being a sixth parameter, para_3 as a real number within 2.8 and 3.9 for the normal healthy mode, within 2.86 and 5.58 for the diabetes pre-stage mode, and within 4.68 and 19.5 for the diabetes mode, para_4 as a real number within 60 and 70 for the empty stomach mode, and within 71 and 80 for the meal after mode, para_5 as a real number within 0.03 and 0.06, para_6 as a real number within 10.211 and 10.519.

10. The cloud system as claimed in claim 8, wherein the output electrode unit comprises a first output electrode and a second output electrode, the input electrode unit comprises a first input electrode, a second input electrode, a third input electrode, and a fourth input electrode, the first output electrode has a ring shape with a central hollow, the second output electrode has a shape of a pattern, the second output electrode is provided in the ring shape of the first output electrode, the first input electrode has a ring shape with a central hollow, each of the second input electrode, the third input electrode, and the fourth input electrode has a shape of a pattern, the second input electrode and the third input electrode are provided in the ring shape of the first input electrode, the first input electrode, the second input electrode and the third input electrode are not in contact with each other, the fourth input electrode is provided in the ring shape of the first output electrode, the fourth input electrode, the first output electrode, and the second output electrode are not in contact with each other, each of a size of the ring shape of the first output electrode and a size of the ring shape of the first input electrode is equal to or larger than a contact area of a finger tip of the finger of the user in contact with the input electrode unit or the output electrode unit, and the blood glucose calculation process performed by the mobile electronic device comprises:
sampling and collecting the sensing signal;
averaging eight to twenty successive sensing signals to calculate an arithmetic mean signal and comparing the arithmetic mean signal with a preset noise threshold until the arithmetic mean signal is not larger than the preset noise threshold, the arithmetic mean signal not larger than the preset noise threshold served as an effective sensing signal, the preset noise threshold being a real number within 300 and 500;
dividing the effective sensing signal into a first finger signal and a second finger signal, the first finger signal served as a signal from a first finger of the user in contact with the first input electrode, the second input electrode, and the third input electrode, the second finger signal served as a signal from a second finger of the user in contact with the first output electrode, the second output electrode, and the forth input electrode, the first finger is a thumb or a forefinger of a right hand of the user, and the second finger is a thumb or a forefinger of a left hand of the user, or alternatively, the first finger is the thumb or the forefinger of the left hand, and the second finger is the thumb or the forefinger of the right hand;
calculating a first finger feedback signal based on the first finger signal by an equation specified by A1_ratio=P1*A1_m_ave+P2, A1_ratio indicating the first finger feedback signal, P1 being a first parameter as a real number within 0.05 and 0.08, P2 being a second parameter as a real number within 21.05 and 35.34, A1_m_Ave being an average of A1_m, A1_m being A1_ave not larger than a value specified as 600 to 1500 and served as a stable feedback signal out of an extreme range, A1_ave being a value of 100 average signals, each average signal being an average of 10 successive first finger signals;
calculating a second finger feedback signal based on the second finger signal by A2_m_Ave, A2_m_Ave being an average of A2_m, A2_m being A2_ave not larger than a value specified as 900 to 1800 and served as a stable feedback signal out of an extreme range, A2_ave being a value of 100 average signals, each average signal being an average of 10 successive second finger signals; and
calculating blood glucose based on the first finger feedback signal and the second finger feedback signal, incorporating blood glucose into the blood glucose information, and the application controlling the operation frame displayed on the display screen to provide an operation mode to the user to select, the operation mode comprising at least one of an empty stomach mode, a meal after mode, a normal healthy mode, a diabetes pre-stage mode, and a diabetes mode, the blood glucose indicated by GLU and calculated by an equation as GLU=P3*(A2_m_ave/P4)−P5)*(((P6−A1_ratio)/10.238)−P5)*P7, P3 being a third parameter, P4 being a fourth parameter, P5 being a fifth parameter, P6 being a sixth parameter, P7 being a seventh parameter, P3 as a real number within 0.8 and 1.0 for the normal healthy mode, within 1.1 and 1.5 for the diabetes pre-stage mode, and within 1.8 and 5.0 for the diabetes mode, P4 as a real number within 210 and 220 for the empty stomach mode, and within 200 and 210 for the meal after mode, P5 as a real number within 0.03 and 0.06, P6 as a real number within 60 and 70 for the empty stomach mode, and within 71 and 80 for the meal after mode, P7 as a real number indicating percentage within 3%-15%.
